(19) United States
(12) Patent Application Publication
(10) Pub. No.: US 2026/0331307 A1
(43) Pub. Date: Oct. 22, 2026

(54) NON-PROFESSIONAL HEALTHCARE, FIELD TREATMENT, AND DOCUMENTATION OF CONDITION AND PRE-HOSPITAL INTAKE COORDINATION TO INCLUDE MASS CASUALTY TRIAGE AND PATIENT INTAKE COORDINATION

(71) Applicants: Christian Ramsey, Colorado Springs, CO (US); Alvaro Rodriguez, Colorado Springs, CO (US); Braxton Toler, Colorado Springs, CO (US); Lincoln Ramsey, Colorado Springs, CO (US)

(72) Inventors: Christian Ramsey, Colorado Springs, CO (US); Alvaro Rodriguez, Colorado Springs, CO (US); Braxton Toler, Colorado Springs, CO (US); Lincoln Ramsey, Colorado Springs, CO (US)

(21) Appl. No.: 19/186,052

(22) Filed: Apr. 22, 2025

(51) Int. Cl. G16H 80/00; G06Q 50/20; G06T 7/00; G16H 10/60; G16H 40/20; G16H 70/20; H04W 4/90

(52) U.S. Cl. CPC G16H 80/00; G06Q 50/20; G06T 7/0012; G16H 10/60; G16H 40/20; G16H 70/20; H04W 4/90; G06T 2207/10016; G06T 2207/30004

(57) ABSTRACT

A system may include a computer-based application program including an image and video capture module configured to document injuries through photos and video recordings; an analysis module configured to assess injury severity based on predefined Tier 1 Evidence-based Medicine protocols and county-specific emergency treatment guidelines; a treatment recommendation module configured to generate field treatment instructions tailored for untrained; users based on injury data; a communication module configured to transmit injury documentation and treatment recommendations to emergency responders, pre-hospital intake coordinators, and healthcare facilities; and an offline data transfer module configured to facilitate peer-to-peer communication between computing devices in the absence of internet connectivity, wherein the system enables non-professional users to document injuries, receive treatment guidance, and share medical data with emergency responders while ensuring compliance with HIPAA regulations.

### TECHNICAL FIELD

The embodiments generally relate to systems and methods for non-professional emergency response, injury documentation, and pre-hospital intake coordination.

### BACKGROUND

Emergency medical care often relies on trained professionals such as emergency medical technicians (EMTs) and paramedics to assess, document, and treat injuries in the field. However, in many cases, untrained individuals, such as bystanders or disaster volunteers, are the first to respond to a medical emergency and/or have not been trained to use first aid or medical devices and furthermore do not know what medications may or may not be used safely for a victim awaiting professional treatment. Traditional emergency response systems do not provide structured guidance for non-professionals to administer first aid, document injuries, or relay critical information to emergency medical personnel. As a result, valuable time is often lost in the moments between an injury occurring and professional medical intervention.

Existing solutions for pre-hospital documentation and treatment coordination primarily focus on trained professionals and require network connectivity to function effectively. Many rely on cloud-based storage, mobile applications with internet access, or integration with emergency dispatch systems. However, these systems often do not account for situations where no professional responders are immediately available or where internet connectivity is unavailable, such as in remote locations or disaster zones. Systems also do not account for any pre-existing conditions or medications that may be known by an individual and may have a material effect on normal field treatment of first aid.

Additionally, while some applications provide basic first-aid instructions, they are often presented in text-heavy formats, requiring literacy, language proficiency, and medical knowledge. These systems lack intuitive, visual step-by-step treatment guidance designed specifically for untrained users. Furthermore, they do not address mass casualty event (MCE) coordination, where multiple injured individuals must be triaged and transported efficiently to appropriate care facilities. In some cases, untrained or inexperienced individuals or volunteers need guidance in correct use or first aid or medical devices when no instructions or professional guidance is otherwise available. In all cases or emergency treatment at the scene of an injury, recording injury evidence may be further useful for successful prosecution of any suspected criminal activity.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

A system and method for non-professional emergency response, injury documentation, and pre-hospital intake coordination provide a structured and intuitive platform to guide untrained individuals in medical emergencies. The system enables users to document injuries using photos and videos, assess injury severity based on predefined Tier 1 Evidence-based Medicine protocols, and generate step-by-step treatment recommendations tailored for non-professionals. The system further facilitates communication with emergency responders, hospitals, and pre-hospital intake coordinators to streamline patient transport and care.

The system includes an image and video capture module for documenting injuries, an analysis module for assessing injury severity, and a treatment recommendation module that provides visual and step-by-step instructions to guide non-professional users. A communication module allows secure transmission of injury documentation and triage assessments to emergency responders, while an offline data transfer module enables device-to-device communication when internet connectivity is unavailable.

The system further supports mass casualty event coordination by allowing county emergency service coordinators to aggregate and prioritize injury reports, ensuring efficient patient transport and hospital intake management. It also includes a training mode to allow users to simulate emergency response scenarios and practice injury documentation, triage assessment, and coordination with emergency services.

By integrating intuitive, highly visual treatment guidance with secure documentation and emergency coordination features, the system addresses critical gaps in existing emergency response solutions. The platform ensures that untrained individuals can effectively assist in medical emergencies, improving patient outcomes while maintaining compliance with HIPAA and other regulatory requirements.

In emergency situations, the system provides critical real-time support to untrained users, enabling them to administer appropriate first aid, document injuries accurately, and communicate effectively with professional responders. By leveraging offline data transfer capabilities, the system ensures that emergency coordination remains functional even in network-compromised environments, making it an invaluable tool for disaster response and remote medical aid.

Other illustrative variations within the scope of the invention will become apparent from the detailed description provided hereinafter. The detailed description and enumerated variations, while disclosing optional variations, are intended for purposes of illustration only and are not intended to limit the scope of the invention.

The system may include functionality for recording pre-existing medical conditions of individuals or small groups, enabling users to input and store chronic diagnoses, allergies, medications, or relevant surgical history within a secure profile. This data may be stored in a structured database and accessed through a user authentication interface when injury documentation is initiated. During triage, the system may reference these records to adjust severity assessments and avoid contraindicated recommendations. This is particularly useful in group settings such as households, sports teams, or care facilities, where shared profiles may be managed under a single administrative account.

The system may operate in modes designed for individual or small group environments, such as field teams, classrooms, or families. A single smart device may support access to multiple user profiles, allowing individualized medical records to be maintained while sharing access to common features such as communication settings, offline synchronization, and data storage. Each profile may retain personal health data, while the system coordinates group-wide response logistics and documentation.

The system may be configured to interface with wearable health monitors or implanted medical devices to automatically retrieve real-time physiological data, such as heart rate, blood oxygen levels, glucose readings, or cardiac telemetry. These inputs may be received via secure application interfaces or local wireless communication and integrated into injury assessment or treatment decision logic. Physiological data from such devices may also be archived for future review or transmitted to emergency medical personnel during a critical event.

The image processing capabilities of the system may be enhanced to include recognition of first aid and medical equipment, allowing it to monitor proper device use during emergency care. For example, the system may verify that a tourniquet is placed in the correct position or that a defibrillator is used in accordance with manufacturer guidelines. Image classification techniques may be applied to identify procedural errors, with prompts delivered to the user to support corrective action. This ensures that the guidance provided by the system is not only procedural but responsive to the user's actual execution.

In certain configurations, the system may deliver treatment instructions using augmented reality overlays presented on a mobile device screen. These overlays may guide users through wound care or other procedures by superimposing visual instructions directly onto a live image feed. Visual markers may highlight the precise placement of bandages, splints, or other medical tools, offering real-time assistance to individuals without formal training and increasing the accuracy of treatment administered in the field.

The system may also support thermal and spectral image capture for enhanced injury evaluation. When used with compatible devices, thermal imaging may detect elevated skin temperatures indicative of infection or inflammation, while spectral imaging may reveal subcutaneous bruising, compromised tissue, or embedded materials. These data sources may supplement standard image analysis to deliver a more complete assessment of injury severity and inform more precise treatment instructions.

In environments where visual access to the device is limited, the system may operate in an audio-only mode to deliver decision support. Treatment instructions may be converted into spoken guidance using integrated text-to-speech tools, allowing users to receive step-by-step support without the need to interact with the screen. This capability may be especially helpful in dark or hands-busy conditions, or for users with limited literacy or visual impairments, ensuring accessibility without compromising responsiveness.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram, according to some embodiments;

FIG. 2 illustrates an application program and modules in communication with the computing system, according to some embodiments;

FIG. 3 illustrates a block diagram of a non-professional healthcare, field treatment, and documentation of condition and pre-hospital intake coordination to include mass casualty triage and patient intake coordination, according to some embodiments;

FIG. 4 illustrates a block diagram of a non-professional healthcare, field treatment, and documentation of condition and pre-hospital intake coordination to include mass casualty triage and patient intake coordination, according to some embodiments;

FIG. 5 illustrates a flowchart of practicing a method a non-professional healthcare, field treatment, and documentation of condition and pre-hospital intake coordination to include mass casualty triage and patient intake coordination, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The system comprises at least one computing device in operable communication with a network and an application server hosting an application program. The application program includes an image and video capture module that enables users to document injuries through photos and video recordings. The image and video capture module may access a device’s built-in camera, utilize image stabilization algorithms to enhance clarity, and employ compression techniques to optimize storage and transmission efficiency. The module may impose limitations on video recording duration and the number of videos per incident, preventing excessive data storage while ensuring sufficient documentation for injury assessment.

An analysis module processes the captured images or videos to assess injury severity. The analysis module may utilize computer vision techniques, such as edge detection and color-based segmentation, to identify wound characteristics, including depth, size, and bleeding intensity. The module may also compare captured images against a database of pre-classified medical conditions using machine learning models trained on annotated injury datasets. The analysis results may be cross-referenced with predefined Tier 1 Evidence-based Medicine protocols and county-specific emergency treatment guidelines to determine appropriate treatment recommendations. The module may also adapt treatment recommendations based on user location, available local emergency medical services, and jurisdictional treatment protocols, utilizing geospatial data for accurate regional classification.

A treatment recommendation module generates step-by-step field treatment instructions tailored for untrained users based on the analyzed injury data. These instructions may be highly visual, using augmented reality overlays or animated instructional guides to depict proper wound care techniques. The module may utilize a decision tree algorithm to dynamically adjust recommendations based on injury severity and available resources. The instructions may be structured for ease of understanding by individuals with no formal medical training, providing real-time interactive guidance that ensures users administer appropriate first aid.

A communication module facilitates the secure transmission of injury documentation and treatment recommendations to emergency responders, pre-hospital intake coordinators, and healthcare facilities. The communication module may employ encryption techniques, such as AES-256 encryption, to protect patient data. The module may integrate with emergency dispatch systems through standardized protocols like HL7 or FHIR, ensuring interoperability with existing medical infrastructure. The communication module may also include an emergency alert function that automatically transmits injury documentation and triage levels to emergency response personnel when a critical condition threshold is met. Additionally, the module may support multimedia messaging to send annotated images, timestamps, and patient status updates to emergency medical teams.

The system includes an offline data transfer module that enables peer-to-peer communication between computing devices in the absence of internet connectivity. This module may utilize Bluetooth Low Energy (BLE) or Wi-Fi Direct to synchronize medical data between devices, enabling secure transmission during network outages or disaster scenarios. The module may employ cryptographic hash functions to verify data integrity and prevent tampering. The offline data transfer module may also implement a store-and-forward mechanism, allowing messages and images to be queued and automatically transmitted when network connectivity is restored, ensuring continuity of care.

The system may further incorporate mass casualty event coordination features that allow county emergency service coordinators to aggregate and prioritize injury reports, assisting in efficient patient transport and hospital intake management. The system may provide real-time triage mapping, displaying patient locations and severity levels on a geographical interface. Additionally, a training mode may be provided, allowing users to simulate emergency response scenarios, practice injury documentation, triage assessment, and coordination with emergency services. The training module may leverage historical incident data to generate realistic emergency scenarios for practice sessions.

The system may support encrypted user medical profiles that store pre-existing conditions, medications, and emergency contacts. These encrypted profiles enable responders to access critical patient history during emergency situations while ensuring compliance with HIPAA regulations and other data privacy standards. The module may implement role-based access controls, ensuring only authorized personnel can retrieve and modify patient records. Additionally, the module may integrate biometric authentication for secure profile access.

The communication module may also interface with telehealth services, allowing remote medical professionals to provide real-time guidance to untrained users administering emergency care. This feature enhances the system’s ability to support medical intervention in remote or resource-limited environments by enabling live consultations via low-bandwidth video streaming or asynchronous messaging. The module may incorporate automated speech-to-text transcription and language translation services to facilitate multilingual communication between users and remote medical professionals.

Various implementations of the invention involve the technical field of computer-based non-professional emergency response, injury documentation, and pre-hospital intake coordination including an image and video capture module configured to document injuries through photos and video recordings; an analysis module configured to assess injury severity based on predefined Tier 1 Evidence-based Medicine protocols and county-specific emergency treatment guidelines; a treatment recommendation module configured to generate field treatment instructions tailored for untrained; users based on injury data; a communication module configured to transmit injury documentation and treatment recommendations to emergency responders, pre-hospital intake coordinators, and healthcare facilities; and an offline data transfer module configured to facilitate peer-to-peer communication between computing devices in the absence of internet connectivity, wherein the system enables non-professional users to document injuries, receive treatment guidance, and share medical data with emergency responders while ensuring compliance with HIPAA regulations and are therefore necessarily rooted in computer technology. For example, the aforementioned steps are inherently computer-based and cannot be performed in the human mind. Additionally, the steps of the present invention would be impossible to accomplish on pen and paper due to the volume of data being communicated and received over a network in real-time. In particular, the speed at which the steps of the present invention occur to effectuate the disclosed method, system, or product would involve large-scale, continuous wireless communication of such data. That is, the steps of the present method, system, or product are impossible to accomplish on pen and paper, cannot be accomplished as a method of organizing human activity, and amount to significantly more than merely gathering, analyzing, and outputting data.

Implementations of the present invention include implementing (executing, running, or deploying) one or more artificial intelligence models on a computing device wherein the computing device executes the artificial intelligence model’s algorithms and mathematical functions on computer hardware using machine learning libraries. The computing device implements the artificial intelligence model when it performs tasks like training, making predictions, applying the model to data, decision-making, classification, or generating outputs based on inputs. In particular, the speed at which an artificial intelligence model analyzes and transforms data to effectuate the disclosed method, system, or product would involve large-scale, continuous transformation of such data. As such, the present invention would be impossible to accomplish on pen and paper or in the human mind due to the volume of data being analyzed and transformed by the artificial intelligence model.

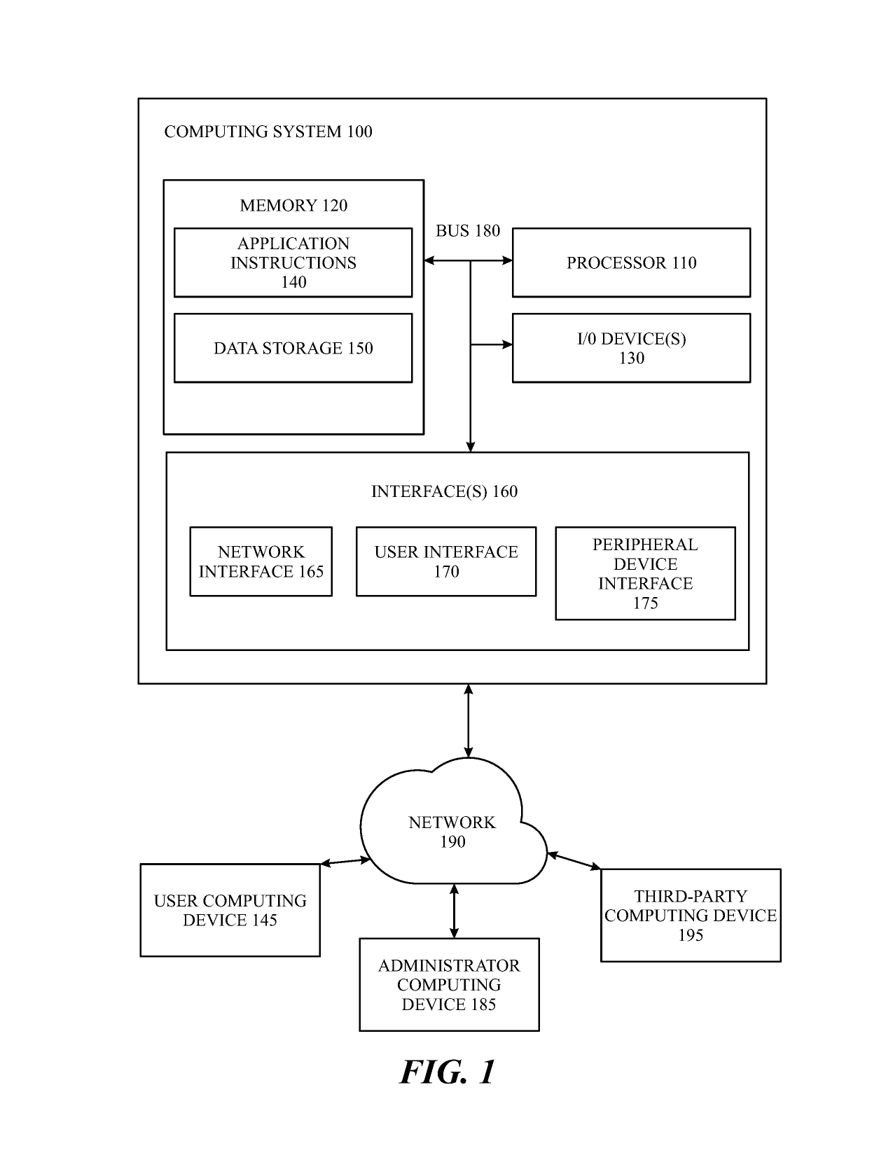

FIG. 1 illustrates an example of a computer system 100 that may be utilized to execute various procedures, including the processes described herein. The computer system 100 comprises a standalone computer or mobile computing device, a mainframe computer system, a workstation, a networked computer or processing device, a desktop computer, a laptop, implanted device or the like. The computer system 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive) or be a bio-implanted device.

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system, or linked computer devices 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 140, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user’s computer, partly on the user’s computer, as a stand-alone software package, partly on the user’s computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user’s computer.

In some embodiments, the computer system 100 may include a user computing device 145, an administrator computing device 185 and a third-party computing device including wearable and implanted devices or non-networked third party devices that can be observed by the computer system 195 each in communication via the network 190. The user computing device 145 may be utilized by a user to interact with the various functionalities of the system. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

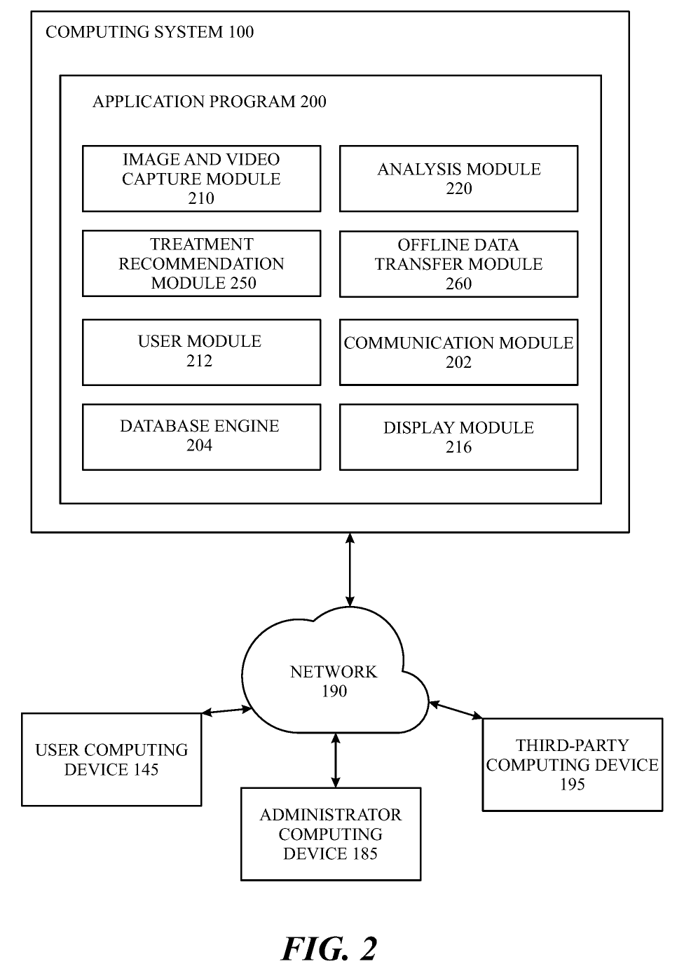

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of an image and video capture module 210, an analysis module 220, a treatment recommendation module 230, an offline data transfer module 240, a communication module 202, a database engine 204, a user module 212, and a display module 216.

In some embodiments, the image and video capture module 210 is configured to enable users to document injuries through photos and video recordings using a computing device's (through a camera, infrared camera or other light filtering device available to be networked or observed by the computer device) built-in camera. Within the context of the system, this module facilitates real-time capture of visual data to assist in assessing injury severity and generating treatment recommendations for non-professional users. The image and video capture module 210 may incorporate image stabilization algorithms to improve clarity in dynamic environments, ensuring that injury documentation remains clear and useful for analysis. To optimize storage and transmission efficiency, the image and video capture module 210 may compress image and video files while maintaining diagnostic quality. In some embodiments, the image and video capture module 210 may impose predefined limitations on video recording duration and the number of video captures per incident, ensuring that essential injury documentation is captured without excessive data usage. Additionally, metadata such as timestamps, GPS coordinates, and environmental conditions may be automatically associated with each captured image or video to provide context for emergency responders and medical professionals. The image and video capture module 210 may also include real-time annotation tools, allowing users to highlight areas of concern within the captured images before transmission. In conjunction with the analysis module 220, the image and video capture module 210 may integrate computer vision techniques, such as edge detection and color-based segmentation, to pre-process images and identify potential indicators of injury severity, such as bruising, bleeding, or swelling. These features collectively ensure that injury documentation is both accessible to non-professional users and actionable for medical personnel receiving the transmitted data.

In some embodiments, the analysis module 220 is configured to process captured images and videos to assess injury severity and generate relevant treatment recommendations based on predefined medical guidelines. Within the context of the system, the analysis module 220 enables non-professional users to receive structured guidance on injury management by leveraging computer vision and machine learning (ML) techniques to analyze visual data. The analysis module 220 may utilize edge detection, color-based segmentation, and object recognition algorithms to identify key wound characteristics, such as depth, size, and bleeding intensity. These characteristics may be compared against a database of pre-classified medical conditions, allowing the analysis module 220 to categorize injuries based on historical medical data. In some embodiments, the analysis module 220 may apply machine learning models trained on annotated injury datasets to enhance injury classification accuracy and refine treatment recommendations dynamically. To ensure alignment with established medical practices, the analysis module 220 may cross-reference injury data with Tier 1 Evidence-based Medicine protocols and county-specific emergency treatment guidelines. This functionality allows treatment recommendations to be tailored based on geographic location, available emergency medical services, and jurisdictional treatment standards. Additionally, the analysis module 220 may assess environmental factors captured in the image metadata, such as ambient lighting, temperature, and location, to provide further contextual awareness. In some implementations, the analysis module 220 may generate confidence scores for its assessments, enabling users or emergency responders to verify the system’s recommendations before proceeding with treatment. By processing injury documentation in real time, the analysis module 220 ensures that untrained individuals receive accurate and timely assistance in emergency situations, ultimately improving pre-hospital care coordination and reducing response times.

In some embodiments, the treatment recommendation module 230 is configured to generate step-by-step field treatment instructions based on the injury data analyzed by the system. Within the context of the invention, the treatment recommendation module 230 assists non-professional users in administering appropriate first aid by providing highly visual, interactive, and language-independent guidance tailored to the severity and type of injury. The treatment recommendation module 230 may utilize decision tree algorithms to dynamically adjust recommendations based on multiple factors, including the injury’s severity, available medical resources, and user location. For example, if a wound is classified as a deep laceration, the treatment recommendation module 230 may first recommend applying pressure, followed by sterilization and bandaging techniques. If the injury is more severe and requires professional medical intervention, the treatment recommendation module 230 may instruct the user on how to stabilize the injured person while awaiting emergency responders. To enhance accessibility, the treatment recommendation module 230 may present animated instructional guides, augmented reality (AR) overlays, or simplified step-by-step text instructions with visual cues to assist users with limited medical knowledge. These instructions may be formatted for quick comprehension in high-stress emergency situations, allowing users to follow guidance effectively even in chaotic environments. In some embodiments, the treatment recommendation module 230 may integrate jurisdiction-specific emergency medical guidelines, ensuring that the recommended treatment aligns with county-level or national medical protocols. Additionally, if local emergency services provide telehealth support, the treatment recommendation module 230 may interface with live or asynchronous telemedicine consultations, allowing a remote medical professional to override automated recommendations or provide further instructions in real time. By structuring first aid guidance in an interactive and adaptive manner, the treatment recommendation module 230 enables untrained individuals to respond effectively to injuries, bridging the gap between the moment an injury occurs and the arrival of professional medical assistance.

In some embodiments, the offline data transfer module 240 is configured to enable peer-to-peer communication between computing devices when internet connectivity is unavailable. Within the context of the invention, the offline data transfer module 240 ensures that injury documentation, analysis results, and treatment recommendations can be shared among users, emergency responders, and medical personnel even in network-compromised environments, such as disaster zones or remote locations. To achieve this, the module may employ Bluetooth Low Energy (BLE), Wi-Fi Direct, or other short-range wireless communication protocols to establish direct device-to-device connections. These connections allow for the secure transmission of captured images, videos, triage assessments, and treatment instructions, ensuring that emergency response efforts are not disrupted due to lack of cellular or internet service. In some implementations, the offline data transfer module 240 may incorporate a store-and-forward mechanism, allowing data to be queued and automatically transmitted when a network connection is restored. This ensures that emergency response teams, hospitals, and medical databases receive critical patient information as soon as connectivity is available, minimizing delays in care coordination. To maintain data security and integrity, the offline data transfer module 240 may utilize cryptographic hash functions and AES-256 encryption to protect sensitive medical data during offline transmission. Additionally, it may implement data synchronization protocols to prevent duplicate records and ensure that the most up-to-date information is available to all connected devices. In mass casualty scenarios, the offline data transfer module 240 may facilitate distributed triage coordination, allowing multiple users in the field to share patient injury statuses and transport prioritization data with on-site emergency coordinators. This enhances situational awareness and resource allocation in large-scale emergencies. By enabling reliable, encrypted, and autonomous data exchange without reliance on cloud services or external networks, the offline data transfer module 240 ensures that the system remains operational in challenging environments, improving pre-hospital emergency care and coordination.

In some embodiments, the communication module 202 is configured for securely transmitting emergency data to responders and hospitals, ensuring timely medical intervention as well as receiving, processing, and transmitting a user command and/or one or more data streams. In such embodiments, the communication module 202 performs communication functions between various devices, including the user computing device 145 of FIG. 1, the administrator computing device 185 of FIG. 1, and a third-party computing device 195 of FIG. 1. In some embodiments, the communication module 202 is configured to allow one or more users of the system, including a third-party, to communicate with one another. In some embodiments, the communications module 202 is configured to maintain one or more communication sessions with one or more servers, the administrative computing device 185 of FIG. 1, and/or one or more third-party computing device(s) 195 of FIG. 1. In some embodiments, the communication module 202 may allow users and administrators to communicate with one another.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

The user module 212 may store user preferences including the user account information, historical usage data, user personal information, and the like. The user module 212 may facilitate the creation of user's profiles for users, administrators, and others.

In some embodiments, the display module 216 is configured to display one or more graphic user interfaces, including, e.g., one or more user interfaces. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. The display module 216 displays information, notifications, and alerts to the user device which can be viewed and acknowledged by the user.

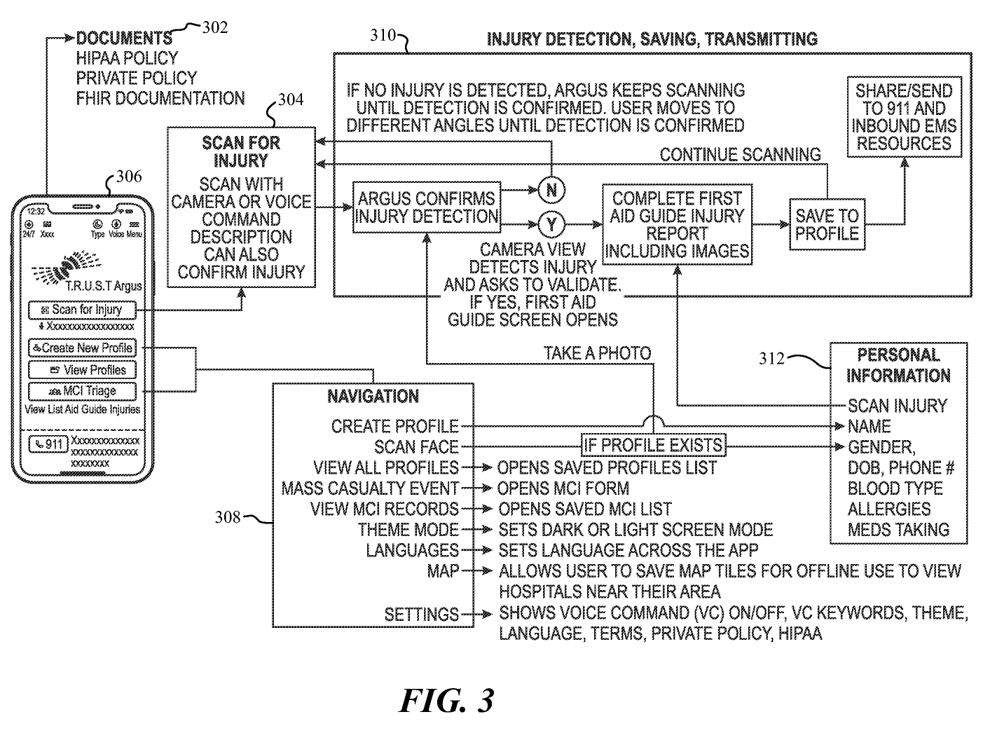

FIG. 3 illustrates the functional architecture of the emergency response system, detailing how various modules interact to facilitate injury documentation, analysis, treatment recommendation, data transfer, and communication in both connected and offline environments. Documents 302 correspond to records generated and managed by the system, which may include injury documentation, analysis results, treatment recommendations, and patient history. Documents 302 may be functionally linked to or stored in the database engine 204, which stores structured data and enables efficient retrieval by authorized system components. The documents 302 may be accessed by the user module 212, which authenticates users and controls role-based access permissions to ensure compliance with regulatory requirements such as HIPAA. A scan for injury 304 function interfaces with the image and video capture module 210 to acquire images and videos of injuries in real-time. The image and video capture module 210 may employ computational photography techniques, such as adaptive exposure control, edge detection, and contrast enhancement, to improve image clarity and diagnostic quality. Once an image or video is captured, it is transmitted to the analysis module 220 for further processing. This process may include metadata tagging, where the system appends timestamps, GPS coordinates, and environmental data to each record for contextual awareness. The smart device 306 component represents mobile computing devices, such as smartphones and tablets, that serve as the primary interface for system users. The display module 216 renders a graphical user interface (GUI) on the smart device, presenting users with interactive prompts, real-time feedback, and step-by-step guidance for documenting injuries. The smart device 306 facilitates user interaction with the system’s core functionalities, including capturing media, reviewing analysis results, and communicating with emergency responders. Navigation 308 functionality integrates geolocation data with system recommendations, assisting users in determining the best course of action during emergency scenarios. The system may utilize GPS, cellular triangulation, or Wi-Fi positioning to provide location-based services. Navigation prompts may be generated by the treatment recommendation module 230, which factors in proximity to medical facilities, estimated travel time, and available emergency services when providing recommendations. For instance, if an injury is classified as severe, the module may recommend the nearest trauma center and display turn-by-turn navigation through the display module 216. Injury detection 310 functionality is driven by the analysis module 220, which employs computer vision algorithms and machine learning models to assess injury severity. The module may use feature extraction techniques, such as convolutional neural networks (CNNs), to identify wound patterns and classify injuries based on severity. The injury classification results are then cross-referenced with predefined Tier 1 Evidence-Based Medicine protocols stored in the database engine 204. Once classified, the injury data is relayed to the treatment recommendation module 230, which formulates a corresponding treatment plan. The treatment recommendation module 230 provides step-by-step first-aid instructions tailored to the analyzed injury data. Recommendations may be displayed as textual descriptions, augmented reality (AR) overlays, or interactive visual guides. These recommendations are rendered by the display module 216 on the user’s smart device 306, ensuring accessibility even for users with limited medical knowledge. The module adapts instructions based on factors such as injury severity, available first-aid supplies, and user location. If a network connection is available, the communication module 202 may escalate critical cases by transmitting the treatment recommendations to emergency responders. The offline data transfer module 240 ensures system functionality in environments with limited or no internet connectivity. If a network connection is unavailable, the module facilitates peer-to-peer (P2P) data exchange via Bluetooth Low Energy (BLE) or Wi-Fi Direct. This capability allows injury data, treatment recommendations, and analysis results to be synchronized between multiple smart devices 306, ensuring that responders in the field remain informed. The offline module implements store-and-forward messaging, allowing data to be queued and transmitted once connectivity is restored. Personal information 312 may include patient-specific data, including medical history, allergies, and emergency contacts. This data is securely stored within the database engine 204 and is accessible through the user module 212, which enforces access control policies. When an injury is documented, the system may cross-reference the captured data with the patient’s medical history to identify potential complications, such as contraindicated treatments due to pre-existing conditions. The communication module 202 serves as the system’s primary interface for transmitting and receiving critical data. When internet connectivity is available, the module transmits injury reports, analysis results, and treatment recommendations to emergency responders, hospitals, and pre-hospital intake coordinators. The module supports multimodal communication, including secure messaging, voice calls, and real-time video consultations. If the system detects a life-threatening injury, the module can trigger an automated emergency alert, instantly notifying nearby responders and dispatch centers.

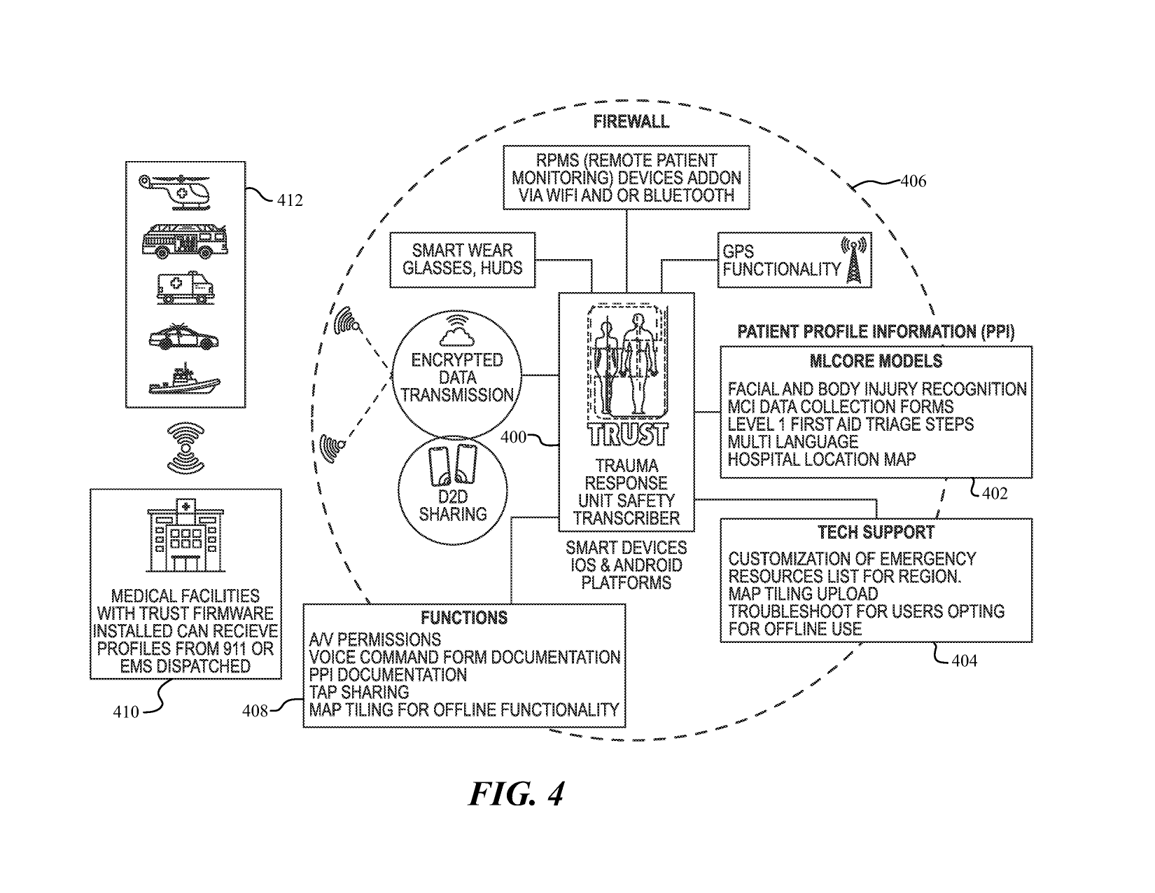

FIG. 4 illustrates the structural and functional components of the system 400, detailing the interactions between machine learning models, security features, emergency response entities, and data management components. The system 400 integrates multiple modules, including the image and video capture module 210, analysis module 220, treatment recommendation module 230, offline data transfer module 240, communication module 202, database engine 204, user module 212, and display module 216 of FIG. 2, to facilitate efficient emergency response operations. ML models 402 interface with the analysis module 220 to process images and videos captured by the image and video capture module 210. These machine learning models may employ convolutional neural networks (CNNs) for wound detection, decision tree algorithms for severity classification, and natural language processing (NLP) techniques to interpret textual descriptions accompanying injury documentation. The ML models 402 continuously improve by incorporating real-world injury data stored in the database engine 204, enhancing accuracy over time. Tech support 404 functionality ensures system stability and operational integrity by monitoring real-time system performance, troubleshooting errors, and providing automated or manual assistance to users. Tech support 404 functionality may be accessible through the user module 212, allowing authorized personnel to diagnose software or hardware issues related to smart devices, network connectivity, and system responsiveness. Firewall 406 serves as a security barrier that protects sensitive medical and personal data processed by the system 400. Firewall 406 regulates incoming and outgoing network traffic between user devices, medical facilities 410, first responders 412, and cloud-based databases. The firewall 406 enforces encryption standards, access control policies, and intrusion detection mechanisms to safeguard confidential health records stored in the database engine 204 and transmitted via the communication module 202. Functions 408 represent the operational capabilities of the system 400, including injury assessment, treatment guidance, emergency alert generation, and secure data transmission. These functions rely on interactions between multiple system modules: the display module 216 presents real-time treatment recommendations generated by the treatment recommendation module 230, while the offline data transfer module 240 ensures seamless peer-to-peer communication when standard network infrastructure is unavailable. Medical facilities 410 receive critical injury data and triage reports via the communication module 202. The system 400 transmits patient information, treatment history, and emergency response updates to hospitals and pre-hospital intake coordinators, ensuring preparedness before the patient arrives. Secure integration with hospital electronic health record (EHR) systems allows medical personnel to access relevant case data without delays. First responders 412 receive automated injury notifications when a critical threshold is met, enabling rapid deployment of emergency medical teams. The communication module 202 facilitates real-time data exchange between first responders 412, medical facilities 410, and system users. When connectivity is unavailable, the offline data transfer module 240 ensures injury documentation and triage data are shared among nearby responders using Bluetooth Low Energy (BLE) or Wi-Fi Direct protocols.

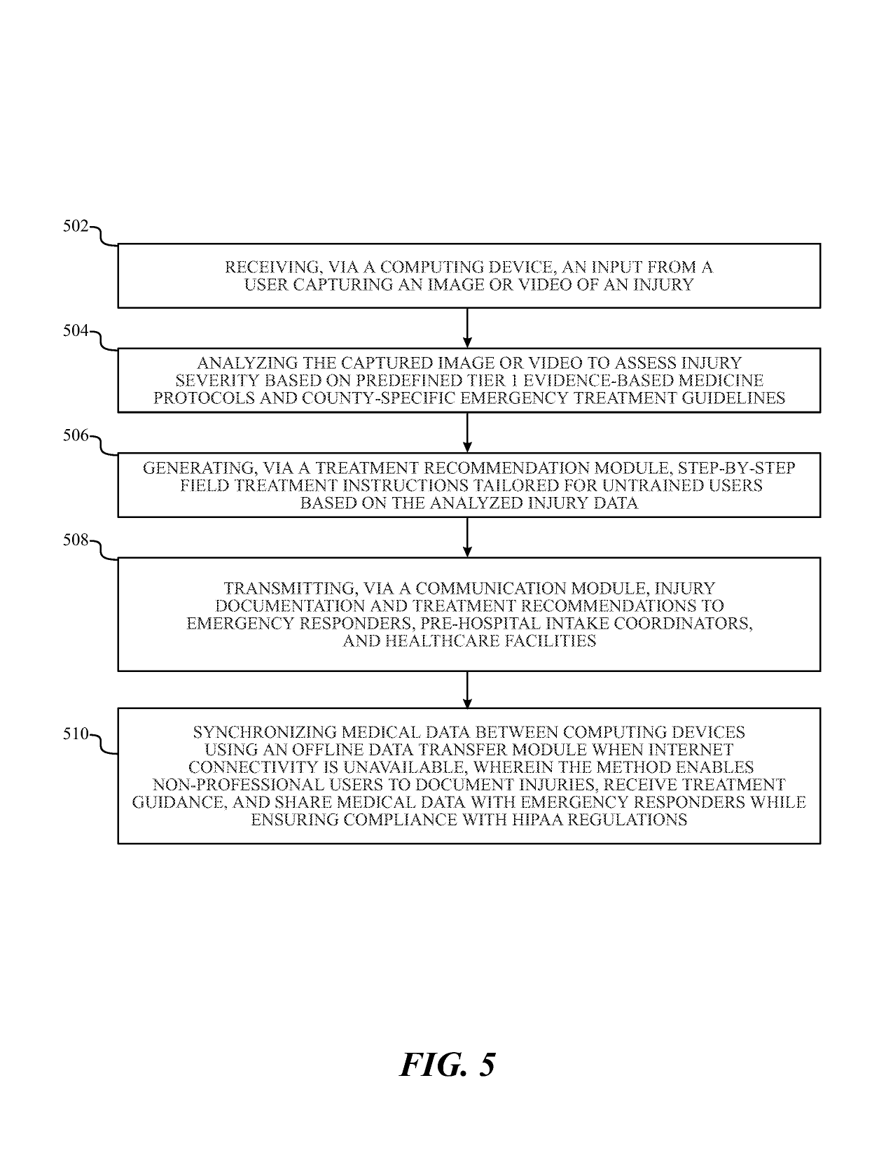

FIG. 5 illustrates a flowchart of practicing a method a non-professional healthcare, field treatment, and documentation of condition and pre-hospital intake coordination to include mass casualty triage and patient intake coordination, according to some embodiments. The method for non-professional emergency response and medical documentation may include, in step 502, receiving, via a computing device, an input from a user capturing an image or video of an injury. Step 504 may include analyzing the captured image or video to assess injury severity based on predefined Tier 1 Evidence-based Medicine protocols and county-specific emergency treatment guidelines. Step 506 may include generating, via a treatment recommendation module, step-by-step field treatment instructions tailored for untrained users based on the analyzed injury data. Step 508 may include transmitting, via a communication module, injury documentation and treatment recommendations to emergency responders, pre-hospital intake coordinators, and healthcare facilities. Step 510 may include synchronizing medical data between computing devices using an offline data transfer module when internet connectivity is unavailable, wherein the method enables non-professional users to document injuries, receive treatment guidance, and share medical data with emergency responders while ensuring compliance with HIPAA regulations.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer’s job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

We claim:

1. A system comprising:
at least one computing device in operable communication with a network;
an application server in operable communication with the at least one computing device over the network, the application server configured to host an application program comprising:
an image and video capture module configured to document injuries through photos and video recordings;
an analysis module configured to assess injury severity based on predefined Tier 1 Evidence-based Medicine protocols and county-specific emergency treatment guidelines;
a treatment recommendation module configured to generate field treatment instructions tailored for untrained users based on injury data;
a communication module configured to transmit injury documentation and treatment recommendations to emergency responders, pre-hospital intake coordinators, and healthcare facilities; and
an offline data transfer module configured to facilitate peer-to-peer communication between computing devices in the absence of internet connectivity, wherein the system enables non-professional users to document injuries, receive treatment guidance, and share medical data with emergency responders while ensuring compliance with HIPAA regulations.

2. The system of claim 1, wherein the image and video capture module is configured to limit video recordings to a predefined maximum duration and restrict the number of videos per incident to optimize data storage and transmission efficiency.

3. The system of claim 1, wherein the analysis module is further configured to adapt treatment recommendations based on user location, available local emergency medical services, and jurisdictional treatment protocols and available first aid and medical devices and any user provided pre-existing health conditions or medications cross referenced to age and at-risk category of a victim.

4. The system of claim 1, wherein the treatment recommendation module provides step-by-step, highly visual, and language-independent instructions designed for untrained users in high-stress environments.

5. The system of claim 1, wherein the communication module includes an emergency alert function that automatically transmits injury documentation and triage levels to emergency response personnel when a critical condition threshold is met.

6. The system of claim 1, wherein the offline data transfer module utilizes Bluetooth Low Energy (BLE) or Wi-Fi Direct to synchronize medical data between devices during network outages or disaster scenarios.

7. The system of claim 1, wherein the system includes a mass casualty event coordination feature that enables county emergency service coordinators to aggregate and prioritize injury reports for efficient patient transport and hospital intake management.

8. The system of claim 1, wherein the system further comprises a training mode that allows users to simulate emergency response scenarios, practicing injury documentation, triage assessment, and coordination with emergency services.

9. The system of claim 1, wherein the system supports encrypted user medical profiles that store pre-existing conditions, medications, and emergency contacts, enabling responders to access critical patient history during emergency situations.

10. The system of claim 1, wherein the communication module is further configured to interface with telehealth services, allowing remote medical professionals to provide real-time guidance to untrained users administering emergency care.

11. A computer-implemented method for non-professional emergency response and medical documentation, comprising:
receiving, via a computing device, an input from a user capturing an image or video of an injury;
analyzing the captured image or video to assess injury severity based on predefined Tier 1 Evidence-based Medicine protocols and county-specific emergency treatment guidelines;
generating, via a treatment recommendation module, step-by-step field treatment instructions tailored for untrained users based on the analyzed injury data;
transmitting, via a communication module, injury documentation and treatment recommendations to emergency responders, pre-hospital intake coordinators, and healthcare facilities; and
synchronizing medical data between computing devices using an offline data transfer module when internet connectivity is unavailable, wherein the method enables non-professional users to document injuries, receive treatment guidance, and share medical data with emergency responders while ensuring compliance with HIPAA regulations.

12. The method of claim 11, wherein the captured image or video is automatically processed to extract injury characteristics and compare them to a database of pre-classified medical conditions.

13. The method of claim 11, wherein the treatment recommendation module adapts instructions based on user location, available local emergency medical services, and jurisdictional treatment protocols.

14. The method of claim 11, wherein the step-by-step treatment instructions are presented using a highly visual, language-independent interface designed for untrained users in high-stress environments.

15. The method of claim 11, wherein an emergency alert function is triggered when a critical condition threshold is detected, automatically notifying emergency response personnel with the injury documentation and triage level.

16. The method of claim 11, wherein the offline data transfer module utilizes Bluetooth Low Energy (BLE) or Wi-Fi Direct to enable peer-to-peer communication of medical data between computing devices during network outages or disaster scenarios.

17. The method of claim 11, wherein a mass casualty event coordination feature aggregates and prioritizes injury reports for efficient patient transport and hospital intake management by county emergency service coordinators.

18. The method of claim 11, wherein a training mode is included to allow users to simulate emergency response scenarios, practice injury documentation, triage assessment, and coordination with emergency services.

19. The method of claim 11, wherein the communication module interfaces with telehealth services, enabling remote medical professionals to provide real-time guidance to untrained users administering emergency care.

20. A software product comprising at least one computer readable storage media having application instructions collectively stored on the at least one computer readable storage media, the application instructions executable to:
receive, via a computing device, an input from a user capturing an image or video of an injury;
analyze the captured image or video to assess injury severity based on predefined Tier 1 Evidence-based Medicine protocols and county-specific emergency treatment guidelines;
generate, via a treatment recommendation module, step-by-step field treatment instructions tailored for untrained users based on the analyzed injury data;
transmit, via a communication module, injury documentation and treatment recommendations to emergency responders, pre-hospital intake coordinators, and healthcare facilities; and
synchronize medical data between computing devices using an offline data transfer module when internet connectivity is unavailable, wherein the method enables non-professional users to document injuries, receive treatment guidance, and share medical data with emergency responders while ensuring compliance with HIPAA regulations.
